(19) United States
(12) Patent Application Publication
(10) Pub. No.: US 2007/0160694 A1
(43) Pub. Date: Jul. 12, 2007

(54) PREVENTIVES OR REMEDIES FOR TUMOR OR HUMAN PAPILLOMAVIRAL DISEASE

(76) Inventors: Nobutaka Suzuki, Ishikawa (JP); Tomihisa Ohta, Ishikawa (JP)

Correspondence Address: BIRCH STEWART KOLASCH & BIRCH, PO BOX 747, FALLS CHURCH, VA, 22040-0747, US

(21) Appl. No.: 11/688,151

(22) Filed: Mar. 19, 2007

Related U.S. Application Data
(62) Division of application No. 10/469,507, filed on Sep. 2, 2003.

(51) Int. Cl. A61K 36/899; A61K 31/525; C12P 1/00

(52) U.S. Cl. 424/750; 514/251; 435/41

(57) ABSTRACT

A composition for foods or medicaments, containing a fermentation product or an enzyme-treated product that is either obtained by fermenting or treating with an enzyme at least one type of component selected from the husks, pellicles and astringent skin of Coix seeds.

### CROSS REFERENCE TO RELATED APPLICATIONS

This application is a Divisional of application Ser. No. 10/469,507 filed on Sep. 02, 2003, which is the national phase application of International Patent Application No. PCT/JP01/01655 having an international filing date of Mar. 2, 2001, which designated the United States of America. The entire contents of all of the above—identified applications are hereby incorporated by reference.

### TECHNICAL FIELD

The present invention relates to a composition for foods or medicaments, which contains a fermentation product or an enzyme-treated product of the husks, pellicles and astringent skin of Coix seeds, or the same compounded with a folic acid or Denshichi ginseng (Panax notoginseng). The pharmaceutical composition has an antineoplastic effect, anti-human papillomaviral disease effect, and an effect against various cutaneous diseases, constipation or the like, so that it is useful for chemoprophylaxis or therapy against tumor, for preventing or treating human papillomaviral diseases such as condyloma acuminatum, verruca vulgaris, adolescent verruca plana, senile verruca or laryngeal papillomatosis, and is useful for preventing or treating various cutaneous diseases or constipation.

### BACKGROUND ART

A grain with the husk [scientific name: seed] of a Coix seed (Job's tears) belonging to the genus Coix of the family Poaceae [scientific name: Coix lachryma-jobi L. var. ma-yuen (Roman.) Staph] is composed of, from the outermost layer to the internal layer, a husk [scientific name: involucre], a pellicle [scientific name: glume, palea and lemma], astringent skin (bran) [scientific name: pericarp], and a grain (sarcocarp) [scientific name: caryopsis]. Many foods such as noodles, confections and the like have been developed using Coix seeds as a raw material. Normally when Coix seeds are taken as foods, their husks, pellicles and astringent skin are removed by threshing, only their grain portions are utilized. Particularly the husk is very hard, so that it cannot be eaten intact. Thus, a Coix seed has been thought to be unsuitable as a material for nutritional supplementary foods or the like. However, as an exception, only the tea of Coix seeds is used by directly decocting the grains with husks. Further, “Yokuinin,” which is described in the “Japanese pharmacopoeia” and is known as a Chinese medicine in Japan, is defined as a grain which is prepared by collecting the grains of Coix seeds with husks, threshing (husks, pellicles and astringent skin are removed) and drying. Therefore, Yokuinin does not contain any husk, pellicle and astringent skin. In the field of foods, a term generally referred to as “Coix seed (Hato-mugi)” often refers to grain after threshing, unless otherwise specified. When a Coix seed contains the husk, pellicle and astringent skin, it is referred to as a Yokuinin with husk, Coix seed with skin, Coix seed with husk or the like. Thus, for example, the one referred to as a Coix seed extract means the extract of threshed Coix seeds, unless otherwise specified. Further, when miso (paste) or vinegar is produced from Coix seeds by fermentation, threshed Coix seeds are used, unless otherwise specified.

The hot water extract of Yokuinin is used medically and clinically in Japan, and the application for insurance reimbursement to the use of Yokuinin for verruca vulgaris and adolescent verruca has been approved. Moreover, when Yokuinin is compounded as a Chinese medicine with other agents, the hot water extract of Yokuinin is also used for this purpose. Clinically, a Yokuinin extract in the form of powder or tablet is used as a pharmaceutical preparation containing a water extract (dry) that has been extracted from Yokuinin. Normally, 6 g of the powder contains 2 g of the water extract (dry), and 18 tablets contain 2 g of the water extract (dry). It is known that the Yokuinin extract is effective against verruca vulgaris and adolescent verruca plana (Yoshitaka YAMADA et al., The Nishinihon Journal of Dermatology, 1993; 55: 106-11, and Kunihide BEPPU et al., Japanese Journal of Medicine and Pharmaceutical Science, 1996; 36: 69-90). However, most of the cases are actually intractable, and difficult to be completely cured. Further, surgical excision, electrocauterization, liquid nitrogen freezing treatment and the like are employed in the therapy for verruca. However, when multiple verrucae occur, they cannot be easily cured in most cases. Furthermore, an anticancer agent such as a bleomycin ointment, a 5-FU ointment or the like may be applied to intractable cases, but there is a concern about its side effect. The advent of a new remedy having high safety has been much awaited.

The effectiveness of Yokuinin against molluscum contagiosum was also reported (Makito NIIMURA et al., skin, 1987; 29: 762-78). However, the disease that is resistant to Yokuinin is often encountered, and it is also difficult to be treated in most cases. Condyloma acuminatum is a disease characterized in that intractable papilloma-like rashes are developed around the vulva or anus due to human papilloma virus, as in verruca vulgaris or the like. The internal use of Yokuinin may show potency (Yoshitaka YAMADA et al., Clinical Dermatology (Hifu Rinsho), 1993; 35: 1020-1). However, Yokuinin is normally thought to have a weak effect against condyloma acuminatum. Thus instead of using Yokuinin, most patients with this disease are treated by electrocauterization or the like, which causes a great deal of pain to the patients. It is thought that the reason why Yokuinin is hardly used for treating viral verruca in countries other than Japan is because of the weak potency against the diseases as described above. Coix seeds are originally safe foods. If a pharmaceutical preparation of Coix seeds having improved potency than that of Yokuinin is developed, the new preparation may be used all over the world.

It has not been completely elucidated that, with what kind of action mechanism, Yokuinin has an effect against viral verruca. Mizoguchi et al., reported that Yokuinin acts on the monocyte-macrophage lineage cells, and then enhances antibody-producing cells through the enhancement of interleukin-1 production (Yasuhiro MIZOGUCHI et al., Journal of Japanese and Chinese Medicine Society (Wakan iyaku-gakkai-shi), 1986; 3: 170-6). Further, Kaneda et al recognized the enhancement of NK cell activity and MHC non-restrictive cytotoxic T cells by the internal use of Yokuinin (Tatsunari KANEDA et al., Clinical Pharmocology, 1992; 40: 179-81). Furthermore, it was reported that cytotoxic T cells are activated by Yokuinin, thereby providing antiviral action (Hidaka Y et al., Biotherapy. 1992; 5: 201-3).

A series of studies on the antineoplastic activity of Yokuinin were started because Ehrlich's ascites carcinoma of mice was suppressed by the acetone extract of Yokuinin after defatting with ether (Muneharu NAKAYAMA et al., Chiba Medical Journal, 1958; 34: 324-5). Then, a fat, Coiexenolide was isolated (Ukita T et al., Chem. Pharma. Bull. 1961; 9: 43-6) and then synthesized (Tanimura A., Chem. Pharma. Bull. 1961; 9: 47-53). This substance was assumed to be the pharmacologically-active ingredient of the antineoplastic activity of Yokuinin. Although supplementary examinations were conducted later for Coiexenolide, it was not confirmed (Tsuneaki NAGAO et al., Lecture summaries of the 103 rd Annual Meeting of the Pharmaceutical Society of Japan, 1983; p 203). It was also suggested that there is a possibility that there are Yokuinin containing Coiexenolide and Yokuinin containing no Coiexenolide (Hiroshi HIKINO, Modern Oriental Medicine, 1988; 9: 51-54). Moreover, there are a report that the active fraction is a free fatty acid mixture (Mitsuhiro NUMATA et al., Lecture Summaries of the 43rd Annual Meeting of The Japanese Cancer Association, 1984; P919) and a report that the active fraction is an unsaturated fatty acid (linolic acid) (Mitsuhiro NUMATA et al., Lecture Summaries of the 46th Annual Meeting of The Japanese Cancer Association, 1987; P1585). However, the activity has not been elucidated yet. Then, it was reported that the oral administration of Yokuinin to rat cholangiocarcinoma suppressed the growth of the carcinoma (Ohai IWANAMI, Journal of OSAKA MEDICAL COLLEGE, 1972; 31:145-161). Further, the carcinostatic action of the methanol extract of Yokuinin (Takuo KOSUGE et al., Yakugaku Zasshi, 1985; 105: 791-5) was reported, and the cytotoxicity against Raji cells (Takao KIJIMA et al., Journal of Herbal Medicine (Shoyaku-shi), 1987; 41: 344-8) was also shown. When carcinogenesis-preventing action was screened for in an Epstein-Barr virus early antigen (EBV-EA) expression test using Raji cells, and then using the Yokuinin showing the prophylactic action, a two-step carcinogenesis suppression test was performed for mouse skin. The number of oncogenesis was decreased in the Yokuinin-administered group and suppression effect was observed. Further, Yokuinin was also observed to have suppression action against carcinogenesis due to UV irradiation when orally administered (Harukuni TOKUDA, Fragrance. Journal. 1995; 8: 94-100). There is a report that when a Yokunin extract was administered to 3 cases of Cervical mild dysplasia (CIN1), the efficacy was seen from 5 months to 1 year after the administration (Enpei CHO et al., The World of Obsterics and Gynecology (Sanfujin-ka-no-sekai), 1999; 51: 111-3). However, since this report includes few cases and does not set any control group, the efficacy is not clearly demonstrated.

Though the antineoplastic effect and the effective ingredient of Yokuinin have been studied as described above, almost nothing about the husks, pellicles and astringent skin of Coix seeds has been studied. Only reported is that the hot water extract and the ether extract of the pericarps and the seed coats of Coix seeds possess cytotoxicity against cultured human T lymphoblastic leukemia cells, cultured human malignant melanoma cells and the like (Kazumasa YASUDA, The Nishinihon Journal of Dermatology, 1983; 45: 203-9., Kazumasa YASUDA et al., Journal of Tokyo Women's Medical University, 1983; 53: 127-131., Kyoko HIRANO et al., The Nishinihon Journal of Dermatology, 1983; 45: 602-8., and Kyoko HIRANO et al., The Nishinihon Journal of Dermatology, 1984; 46: 922-7).

Moreover, the antineoplastic effect of Coix seeds has been reported several times since long before. However, clear clinical effect has not yet been presented so far, so that in actual cases only few doctors in Japan use Yokuinin for cancer treatment, and almost no Yokuinin is used in Europe and the United States.

There has long been a habit of eating fermented foods. Producing foods by fermenting Coix seeds has also been devised. For example, a way of threshing is disclosed in the method for processing Coix seeds (JP Patent Publication (Kokai) No. 5-31380), and the use of only buds germinating from Coix seeds is disclosed in the invention of coixol-containing food (JP Patent Publication (Kokai) No. 6-22726). However in the invention of JP Patent Publication (Kokai) No. 6-22726, the husk, pellicle and astringent skin are not used. The method for extracting Coix seeds, Coix seed extract obtained by the extraction method, and the product for improving skin containing as an active ingredient the Coix seed extract (JP Patent Publication (Kokai) No. 7-274914) discloses in the examples Coix seeds whose epicarps have been removed, but does not disclose any husk, pellicle and astringent skin. The invention of JP Patent Publication (Kokai) No. 7-274914 is a method for extracting a Coix seed extract by adding at least one of a starch-degrading enzyme, proteinase, Aspergillus, and a polysaccharide-degrading microorganism to Coix seeds for reaction to occur. However, such a method using the starch-degrading enzyme or the like for Coix seeds has already been disclosed in the text of the invention of food and drink produced by lactic fermentation of Coix seeds and the production method thereof (JP Patent Publication (Kokai) No. 57-5151), so that this is a known method. Furthermore, the method using proteinase has already been made to be known by the invention of the production method of a physiologically active peptide composition (Japanese Patent No. 3108059). Moreover, the invention of a method using Aspergillus or a polysaccharide-degrading microorganism is also a known method because such the invention has already been disclosed in the above JP Patent Publication (Kokai) No. 57-5151, “Coix seed sauce, its moromi and koji” (JP Patent Publication (Kokai) No. 57-48947), “Method for producing natto(fermented soybeans) containing Coix seeds” (JP Patent Publication (Kokai) No. 58-8826), “Method for producing Coix seed liquor” (JP Patent Publication (Kokai) No. 59-51785, and the like. In addition, JP Patent Publication (Kokai) No. 7-274914 does not disclose that various enzymes are allowed to act on the husks, pellicles and astringent skin of Coix seeds, the fermentation method or the efficacy thereof. In the invention of “Enriched Coix seeds” (JP Patent Publication (Kokai) No. 8-39), a culture medium containing threshed and braised Coix seeds as a principal ingredient is used. The invention of “Anti-allergy agent, antireleaser for chemical mediators, and anti-allergy cosmetic materials, pharmaceutical products and foods that contain the same” (JP Patent Publication (Kokai) No. 10-120583) discloses seeds from which the seed coats of Coix seeds have been removed, but does not disclose the husk. The embodiment of the invention of “External skin preparation” (JP Patent Publication (Kokai) No. 2000-119155) discloses “Coix seed (Yokuinin),” but does not specify the husk, nor the extraction method. “Tyrosinase generation depressant and external skin preparation containing the same” (JP Patent Publication (Kokai) No. 2000-256131) is an external preparation containing a Yokuinin extract mixed therein. The invention of “External skin preparation” (JP Patent Publication (Kokai) No. 2000-319157) specifies that Yokuinin, from which the seed coat is removed, is mainly used. The invention of “External skin preparation” (JP Patent Publication (Kokai) No. 2000-327552) discloses a Coix seed extract, but does not specify the husk, pellicle nor astringent skin. The invention of “External skin preparation” (JP Patent Publication (Kokai) No. 2000-44481) specifies that Coix seeds are extracted using water or an aqueous organic solvent, but does not disclose the husk, pellicle nor astringent skin. Further, there are inventions of “Food and drink produced by lactic fermentation and the production method thereof” (JP Patent Publication (Kokai) No. 52-92662), “Coix seed sauce” (JP Patent Publication (Kokai) No. 55-96074), “Production method of fermented Coix seed” (JP Patent Publication (Kokai) No. 55-54868), “Production method of natto(fermented soybeans) containing Coix seeds” (JP Patent Publication (Kokai) Nos. 58-8826 and 58-31905), “Production method of wart-off external preparation containing Yokuinin as the principal ingredient” (JP Patent Publication (Kokai) No. 5-221870), “Oil-base formulation and the production method thereof” (JP Patent Publication (Kokai) No. 6-9421), “Method for producing flavorings such as miso and soy sauce (JP Patent Publication (Kokai No. 7-236447), “Anti-oxidant composition and the production method thereof” (JP Patent Publication (Kokai) No. 8-103245), “Cell activating agent and external skin preparation compounded with the same” (JP Patent Publication (Kokai) No. 9-77634), and “Coix seed red koji, the production method thereof, α type dry Coix seed red koji, and foods using the same” (JP Patent Publication (Kokai) No. 10-84944). These inventions disclose neither precise examples wherein the husks, pellicles and astringent skin of Coix seeds were fermented nor the importance of the fermentation of the husk, pellicle and astringent skin.

Examples of patent-granted inventions are as follows. The invention of “Production method of Coix seed liquor” (Japanese Patent No. 2631660) does not specify the husk, pellicle nor astringent skin. The pharmaceutical composition for analgesia (Japanese Patent No. 2958198) comprises the thymus of a mammal as a principal ingredient to which a Coix seed extract and the like are added, but this invention does not specify the extract of Coix seeds with husks. The invention of “Pharmaceutical composition for decreasing antinuclear antibodies and pharmaceutical composition for decreasing rheumatoid factors” (Japanese Patent No. 2978432) discloses the thymus as a principal ingredient, and the addition of a Coix seed extract, wherein Coix seeds are extracted by finely cutting dried raw materials of Coix seeds and then extracting with a 30% ethanol solution. The invention of “Production method for physiologically active peptide composition” (Japanese Patent No. 3108059) is a production method which comprises treating polished Coix seed powder with acetic acid and protease, and does not use any husk, pellicle nor astringent skin.

As described above, none of the inventions discloses examples wherein the husks, pellicles and astringent skin of Coix seeds are fermented, nor mentions the medical significance of fermenting or treating, with an enzyme, the husks, pellicles and astringent skin of Coix seeds. The specifications of these inventions contain wrong usages of botanical terms concerning the structure of a Coix seed, and many vague expressions such that any exact part of a Coix seed used in the invention is not specified clearly.

As far as the present inventor has investigated, there are two known products that are produced by fermenting the husks, pellicles and astringent skin of Coix seeds. Nonoyama et al's “Production method for Coix seed vinegar” (JP Patent Publication (Kokai) No. 58-884) is characterized by the germination, drying and roasting of Coix seeds with husks, and the purpose is to confer an excellent flavor to Coix seeds. However, this method does not disclose any medical efficacy. “Brewing method of soy sauce” (Japanese Patent No. 2879618) is for brewing soy sauce using Coix seed grains, and comprises preparing koji using whole soybean- and Coix seed (grain)-compounded raw materials, mixing after the addition of prepared salt water to the koji, fermenting and maturing the mixture to become a mature moromi, pressing out liquids, firing, and then filtering impurities so as to produce soy sauce. This invention is intended for saving the work required to remove the skin of Coix seeds, and is devised to improve defects such that the use of only a part of a Coix seed is a large loss of materials. However, this bulletin does not disclose any medical efficacy of soy sauce.

Patents relating to Coix seeds of countries other than Japan are as follows. “Melanin inhibitor” (U.S. Pat. No. 4,978,523) is a patent relating to the suppression of melanin that is a cinnamic acid derivative, and discloses that the above derivative and a Coix seed are combined, but does not specify the use of the husk, pellicle and astringent skin of a Coix seed. Further, this invention does not disclose a method for extracting Coix seeds. “Fertility drug and method of producing the same” (U.S. Pat. No. 5,023,249) relates to a fertility drug that is extracted from the bran of Coix seeds. “Neutral lipids from endosperm of Job's tears” (U.S. Pat. No. 5,444,089) describes the antineoplastic activity and immunopotentiation of neutral fat that is extracted from the endosperm of the grain of a Coix seed, but does not use any husk, pellicle nor astringent skin. Further the extraction method uses an organic solvent and is different from a method we used herein. The effective substance is also different from that we used herein. “Compositions with analgesic, antipyretic and antiinflammatory properties” (U.S. Pat. No. 5,908,628) is an analgesia, antipyresis and antiinflammatory agent comprising 12 types of component including Coix seeds. The grain of a Coix seed is used herein and the application also differs from that of the present invention. “Roasted soybean hypocotyls and beverage material containing the same” (U.S. Pat. No. 5,972,410) is a patent concerning a beverage comprising roasted soybeans, roasted Coix seeds, and the like, and is not about a fermentation extract or an enzyme extract. Further, the efficacy of the beverage is not described.

All other patents do not disclose any food/extract obtained by fermentation of, food/extract obtained by treatment with an enzyme of the husks, pellicles, astringent skin, and grains of Coix seeds, and the efficacy thereof.

As described above, a number of literature and patents concerning Yokuinin (grain) have been published according to our search. Reports concerning the husks, pellicles and astringent skin of Coix seeds disclose only basic findings about the antineoplastic activity of the hot water extract and the ether extract of the pericarps and the seed coats of Coix seeds as described above. The enzyme food and the extract of the husk, pellicle and astringent skin have not been reported. Further, there is no paper about the fermentation food or the extract of the husk, pellicle and astringent skin. In the field of patent, JP Patent Publication (Kokai) No. 58-884 and Japanese Patent No. 2879618 disclose the same, but do not disclose the efficacy.

Folic acid which are contained in abundance in green vegetables and the like is involved, in co-operation with vitamin B12, in amino acid metabolism and protein synthesis, and specifically in RNA and DNA generation. In this way, folic acid exerts an important function in cell division, cell replication, and tissue growth. Examples of the functions of folic acid include an effect of lowering the risk of the onset of inborn errors such as spondyloschisis and anencephaly, the coenzymatic role involved in neural transmission (involved in dementia, congenital mental retardation, and decreased intelligence quotient), and the involvement in the immune system, particularly in the number and the functions of leucocytes and the formation of erythrocytes.

The relationship between folic acid and cervical dysplasia, carcinoma in situ or progressive cervical carcinoma has already been discussed. Cervical dysplasia consists of mild dysplasia (CIN I), moderate dysplasia (CIN II) and severe dysplasia (CIN III). With higher dysplasia grade, the disease proceeds to carcinoma in situ (carcinoma at early stages: stage 0 of cervical carcinoma, included in CIN III under the CIN classification). CIN is an abbreviation of Cervical Intraepitheal Neoplasia. In addition, CIN I is recently often referred to as low grade SIL, and CIN II and CIN III as high grade SIL. SIL is an abbreviation of Squamous Intraepitheal Lesion.

In 1980, it was indicated that the risk of the onset of cervical carcinoma in oral contraceptive-taking patients may be decreased by the administration of folic acid (Check WA., JAMA. 1980; 244: 633-4). Later, in 1985, it was reported that serum folic acid levels were significantly low in patients with cervical carcinoma (Orr J W Jr et al., Am. J. Obstet. Gynecol. 1985; 153: 775-9). However, it was reported in 1991 that the involvement of serum folic acid levels in causing progressive cervix carcinoma is negative (Potischman N et al., Cancer. Res. 1991; 51: 4785-9). Further, folic acid levels in erythrocytes were measured, so that it was assumed that folic acid has a protective action against CIN (VanEenwyk J et al., Cancer. Epidemiol. Biomarkers. Prev. 1992; 1: 119-24). In another report, folic acid was considered as having a protective action against the onset of cervical dysplasia, but was considered as not having a protective action against the onset of carcinoma in situ and progressive cervical carcinoma (Potischman N., J. Nutr. 1993; 123(2 Suppl): 424-9). Furthermore, the effect of folic acid was examined by actually administering folic acid to humans. Specifically, 5 mg/day of folic acid was administered to 331 patients with koilocytic atypia, mild dysplasia and moderate dysplasia. Six months later, the improving effect was compared among the patients, but no difference was found when compared with the group orally administered with placebo (Childers J M et al., Cancer. Epidemiol. Biomarkers. Prev. 1995; 4: 155-9). Further, 10 mg/day of folic acid was administered to 154 cases of patients with CIN I or CIN II, and then the effect of folic acid was evaluated 6 month later. However, no difference was also found when compared with the group orally administered with placebo (Zarcone R et al., Minerva. Ginecol. 1996; 48: 397-400). A paper published in 1997 is about that folic acid may be involved in the cause of CIN based on the fact that the blood folic acid levels were statistically low in women with CIN-HPV(+) (Kwasniewska A et al., Eur. J. Gynaecol. Oncol. 1997; 18: 526-30). In the next year, 1998, intake levels of folic acid in meals were studied for 7 normal cases, 30 cases with CIN I, 18 cases with CIN II, 13 cases with CIN III and 11 cases with carcinoma in situ (CIS). The result suggested that folic acid is not involved in the carcinogenicity of the uterine cervix (Kantesky P A et al., Nutr. Cancer. 1998; 31: 31-40).

As described above, the relationship between folic acid and the onset of cervical carcinoma is not clearly concluded, and this argument is still in a chaotic state. However at least, a conclusion that has been made so far is that administration of folic acid has no effect on already developed cervical dysplasia, carcinoma in situ or progressive uterine cancer, and lesions are not restored to their normal states. In addition, there have been no report or patent relating to a composition that is prepared by compounding with folic acid the food/extract obtained by fermentation of, or the food/extract obtained by treatment with an enzyme of the husks, pellicles and astringent skin of Coix seeds.

Denshichi ginseng (scientific name: Panax notoginseng) belonging to the Araliaceae family is also referred to as Sanshichi ginseng, produced in Yunnnan, China, and is known to have several active ingredients including saponin. Denshichi ginsengs are foods that have been relatively studied well. Examples of the actions that have been reported in literature include the effect on cardiac functions (Feng P F et al., Chung. Kuo. Chung. Hsi. I. Chieh. Ho. Tsa. Chih. 1997; 17: 714-7, and Huang Y S et al., Burns. 1999; 25: 35-41), antiarrhythmic action (Li X J et al., Yao. Hsueh. Hsueh. Pao. 1988; 23: 168-73, and Liu S et al., Chung. Kuo. Yao. Li. Hsueh. Pao. 1984; 5: 100-3), antihypertensive action (Kwan C Y., Clin. Exp. Pharmacol. Physiol. 1995; 22(Suppl 1): 297-9), action against hemorrhagic shock (Li L X et al., Chung. Kuo. Yao. Li. Hsueh. Pao. 1988; 9: 52-5), action against experimental DIC (Kubo M et al., Yakugaku. Zasshi. 1984; 104: 757-62), action against ischemic encephalopathy (Han J A et al., Chung. Kuo. Chung. Hsi. I. Chieh. Ho. Tsa. Chih. 1996; 16: 506-7, and Jiang K Y et al., Chung. Kuo. Yao. Li. Hsueh. Pao. 1995; 16: 399-402), antilipotropic action (Xu Q et al., Chung. Kuo. Chung. Yao. Tsa. Chih. 1993; 18: 367-8), hypoglycemic action (Gong Y H et al., Yao. Hsueh. Hsueh. Pao. 1991; 26: 81-5), and anti-inflammatory action (Li S H et al., Chung. Kuo. Yao. Li. Hsueh. Pao. 1999; 20: 551-4, and Hao C Q et al., Chung. Kuo. Yao. Li. Hsueh. Pao. 1986; 7: 252-5).

According to tumor-related literature, the immunopotentiation action of polysaccharides obtained from Denshichi ginsengs (Gao H et al., Pharm. Res. 1996; 13: 1196-200), the effect of a Denshichi ginseng-compounded mixed pharmaceutical preparation (name: Hua-sheng-ping) against precancerous lesions (Yu X Y., Chung. Kuo. Chung. Hsi. I. Chieh. Ho. Tsa. Chih. 1993; 13: 147-9), and others are known. In addition, it was reported that a Denshichi ginseng extract suppressed the generation and the growth processes of tumors in a two-step oncogenesis experiment using mouse skin tumors (Konoshima T et al., Biol. Pharm. Bull. 1999; 22: 1150-2).

Examples of patent-related literature are as follows. The invention of skin activator and skin-activating food (JP Patent Publication (Kokai) No. 9-67262) specifies in the text the ginseng used in the invention as Korean ginseng which is not Denshichi ginseng, and does not specify whether the extract is of Coix seeds with husks. Further, the invention does not specify the extraction method. The invention of external skin preparation (JP Patent Publication (Kokai) No. 2000-119155) discloses a ginseng, Panax ginseng and Coix seed (Yokuinin), but does not specify it as Denshichi ginseng. In addition, the Coix seed described refers to “Yokuinin.” Furthermore, it was found that a liver function-activating effect is increased synergistically by a combination of a milk thistle extract, a curcuma extract and a Denshichi ginseng extract, compared with the use of a single extract or the simultaneous use of the two extracts (JP Patent Publication (Kokai) No. 11-189539). In addition, there are inventions of “Healthy beverage” (JP Patent Publication (Kokai) No. 2000-354476), “Healthy food, feed and healthy seasoning mainly comprising tofu and bean curd refuse (JP Patent Publication (Kokai) No. 2000-325044), “Saponin-containing extract and the production method” (JP Patent Publication (Kokai) No. 2000-264896), “Herb-containing coffee and the production method” (JP Patent Publication (Kokai) No. 2000-245348), “Diabetes, hypertension and liver function-improving agent mainly comprising Denshichi ginseng, lychee, and Agaricus, and the production method” (JP Patent Publication (Kokai) No. 2000-143526), “Composition for immunosuppression” (JP Patent Publication (Kokai) No. 11-139979), “Denshichi ginseng-containing propolis” (JP Patent Publication (Kokai) No. 10-215799), “Composition of extract of ginseng belonging to the Araliaceae family” (JP Patent Publication (Kokai) No. 11-290024), “Healthy beverage” (JP Patent Publication (Kokai) No. 9-294572), “Animal and plant herbal medicine” (JP Patent Publication (Kokai) No. 8-92109), “Cosmetic or dermatological composition containing at least one saponin of the ginsenoside type, and its applications, especially for treating the hair” (U.S. Pat. No. 5,663,160), “Use of ginsenoside R.sub.0 or a plant extract containing same to promote collagen synthesis” (U.S. Pat. No. 5,747,538), “Use of triterpensaponins, such as notoginsenoside R1 (NR1) and/or astragaloside (ASIV) for preparing medicaments” (U.S. Pat. No. 5,770,578), “Process for the preparation of metabolites of Ginseng saponins” (U.S. Pat. No. 5,925,537), “Herbal skin regeneration composition and method” (U.S. Pat. No. 6,027,728), “Process for removing impurities from natural product extracts” (U.S. Pat. No. 6,132,726) and the like. However, none of these bulletins contain descriptions concerning a composition which is prepared by compounding Denshichi ginseng with the fermentation product or the enzyme-treated product of the husks, pellicles and astringent skin of Coix seeds.

### DISCLOSURE OF THE INVENTION

The object of the present invention is to provide a pharmaceutical composition or a food containing the fermentation product or the enzyme-treated product of the husks, pellicles and astringent skin of Coix seeds.

As a result of intensive studies to achieve the above objectives, we have succeeded in obtaining a pharmaceutical composition having markedly superior drug efficacy compared with those of conventional pharmaceutical preparations of Coix seeds, by fermenting or treating with enzyme the husks, pellicles and astringent skin of Coix seeds. We have found that the fermentation product or the enzyme-treated product of at least one type of component selected from the husk, pellicle and astringent skin of Coix seed (hereinafter referred to as “husk, pellicle and astringent skin”) has an excellent effect against tumor or human papillomaviral disease, and isolated a novel substance as its major ingredient. Furthermore, we have succeeded in preparing a pharmaceutical composition having a further stronger potency by compounding folic acid and/or Denshichi ginseng into the above pharmaceutical composition, so that we have completed the present invention.

That is, the present invention is as follows.

(A) A composition for foods (excluding vinegar and soy sauce) or medicaments, containing a fermentation product that is obtained by fermenting at least one type of component selected from the husk, pellicle and astringent skin of Coix seed.

The above fermentation is characterized in that it is performed by at least one type of microorganism selected from Aspergillus, lactic acid bacteria and yeast.

(B) A composition for foods or medicaments, containing an enzyme-treated product that is obtained by treating with an enzyme at least one type of component selected from the husk, pellicle and astringent skin of Coix seed.

Examples of an enzyme include at least one type of agent selected from a diastase agent, a Takadiastase agent, an α-amylase agent, a β-amylase agent, a glucoamylase agent, a pectinase agent, a β-glucosidase agent, a cellulase agent, a hemicellulase agent and a xylanase agent.

(C) A composition for foods (excluding vinegar and soy sauce) or medicaments, wherein the composition of (A) above is compounded into a processed product comprising at least one of the hot water extract, the ethanol extract, the fermentation product and the enzyme-treated product of the grains (Yokuinin) of Coix seeds.

(D) A composition for foods or medicaments, wherein the composition of (B) above is compounded into a processed product comprising at least one of the hot water extract, the ethanol extract, the fermentation product and the enzyme-treated product of the grains (Yokuinin) of Coix seeds.

(E) An oligosaccharide fraction, which is extracted from the fermentation or the enzyme-treated product of at least one type of component selected from the husk, pellicle, astringent skin and grain of Coix seed. In the present invention, a composition for foods or medicaments containing the oligosaccharide fraction is also provided.

(F) A composition for foods or medicaments, wherein the above composition or oligosaccharide fraction is compounded with folic acid, Denshichi ginseng or both of them.

(G) A preventive or a remedy against tumors, containing as an active ingredient the above composition or the above oligosaccharide fraction.

Examples of tumors include at least one type of tumor selected from the group consisting of a benign tumor (including a soft part tumor including a giant cell tumor of the tendon sheath, polyp of colon and vocal cord polyp), a precancerous lesion (including cervical dysplasia and tylosis linguae), uterine cancer, vaginal cancer, vulvar cancer, skin cancer, esophageal cancer, oral cavity cancer, gingival cancer, jaw cancer, pharyngeal cancer, vocal cord carcinoma, lung cancer, bladder cancer, thyroid cancer, breast cancer, gastric cancer, colon cancer, pancreatic cancer, renal cancer, ovarian cancer, melanoma, tumor of central nervous system, tumor of peripheral nervous system, mediastinal tumor, hepatic cancer, bile duct cancer, gallbladder cancer, tumor of renal pelvis, ureter cancer, testicular tumor, prostate cancer, choriocarcinoma, fallopian tube cancer, sarcoma, leukemia, erythroleukemia, multiple myeloma, malignant lymphoma and carcinosarcoma. (H) A preventive or a remedy against human papillomaviral diseases, containing as an active ingredient the above composition or the above oligosaccharide fraction.

Examples of human papillomaviral diseases include at least one type of disease selected from the group consisting of condyloma acuminatum, verruca vulgaris, adolescent verruca plana, senile verruca and laryngeal papillomatosis.

(I) A preventive or a remedy, containing as an active ingredient the above composition or the above oligosaccharide fraction, and is against at least one type of condition selected from the group consisting of molluscum contagiosum, follicular keratosis, chloasma, freckle, wrinkle, senile plaque, skin pigmentation, skin roughness, clavus and acne vulgaris.

(J) A preventive or a remedy against constipation or the smell of stool, containing as an active ingredient the above composition or the above oligosaccharide fraction.

(K) A functional food, containing as an active ingredient the above composition or the above oligosaccharide fraction.

The present invention will be described in detail as follows.

Recently, therapies, which lower the carcinogenicity of cells in a precancerous state by administering materials such as natural herbs, foods and vitamins that are very safe for a human body, or synthesized drugs, and therapies, which restore normal cell conditions, have been actively studied. These studies have led to the advent of a new concept of cancer chemophylaxis. For example, prophylaxis using tamoxifen, an anti-estrogen drug, against breast cancer corresponds to this concept. In addition, it has been recently reported that isoretinoin (13-cis retinoic acid), which is one of retinoids (vitamin A derivatives), was effective against leukoplakia (precancerous lesion) in the oral cavity. However, isoretinoin is known to have low safety and cause severe complication, so that a healthy person cannot use it for preventing cancer under present conditions. As far as we have examined, there is no report that human carcinoma in situ (early carcinoma) and dysplasia (precancerous lesion) were actually cured by the use of a substance with high safety. Since cancer becomes extremely resistant against various therapies as it advances, the development of a safe chemical preventive or remedy, with which cancer can be cured before it becomes advanced cancer, is expected. Further, the development of a preventive or a remedy against human papillomaviral diseases, which cause various verrucae and laryngeal papillomatosis, have also been awaited.

The present invention basically relates to the fermentation product and the enzyme-treated product of the husks, pellicles and astringent skin of Coix seeds. In the present invention, a fermentation product that is obtained by fermenting the husks, pellicles and astringent skin of Coix seeds, or an enzyme-treated product that is obtained by treating with an enzyme the husks, pellicles and astringent skin of Coix seeds is prepared. Then, these treated products are used as pharmaceutical compositions, thereby demonstrating the clinical effect against various diseases. In the present invention, the fermentation product includes fermentation extracts, and the enzyme-treated product includes enzyme extracts. Further, the fermentation extracts or the enzyme extracts can be respectively isolated by the methods as described later. Furthermore, the treated-products are compounded with folic acid, Denshichi ginseng or a combination thereof, so that the thus enhanced clinical effect is demonstrated and the product is medically applied. Moreover, an effective substance that is isolated from the husks, pellicles and astringent skin of Coix seeds can also be used in the present invention.

1. Production of the Fermentation Product of the Husks, Pellicles and Astringent Skin of Coix Seed

The fermentation product of the husks, pellicles and astringent skin of Coix seed can be obtained by any existing fermentation method. Specifically, Coix seeds with husks (grains with husks) are washed well with water, and then dried sufficiently. The husks are then lightly ground with a rice-milling machine. After the dehulling treatment, Coix seed grains are separated into undehulled grains and dehulled grains using a sieve with a mesh size of approximately 3.5 (5.6 mm). The undehulled grains are treated again with a rice-milling machine, thereby obtaining the husks, pellicles or astringent skin. At this time, it is necessary to adjust the strength of rice-milling to a degree such that the grains are not broken. 3 to 7 liters of water is added to 1 kg of the husks, pellicles and astringent skin, so that these parts are immersed in water for 1 to 2 hours. The solution containing these parts was gradually heated for 20 to 30 minutes to be boiled, followed by further boiling of 20 to 30 minutes. Subsequently, the solution is concentrated by vacuum concentration or vacuum centrifugation for approximately 5 hours while heating to 40° C. to 50° C. Commercial koji (malt), for example, rice koji that has been prepared by allowing Aspergillus oryzae to directly grow on steamed rice is added, 100 g to 200 g per 1 kg of the husks, pellicles and astringent skin, and then fermented for 48 hours at 30° C. while agitating, followed by sterilization at around 90° C. for approximately 30 minutes. The product is then cooled, and then dried by freeze-drying, vacuum-heating and drying or a spray-dry method, so that the fermentation product of the husks, pellicles and astringent skin of Coix seeds can be obtained. The above fermentation product can be used as a composition for foods or medicaments. The clinical dose is 0.1 to 0.2 g/Kg body weight/day, and 5 g/day of the product is normally used for an adult. The product is preferably administered between meals, such that 2.5 g of the product is administered in the morning and in the evening.

Further, the following processes are performed to obtain fermentation extracts from the fermentation products. Specifically, the above product fermented for 48 hours is sterilized at around 90° C. for approximately 30 minutes, and then filtered by centrifugation. The thus obtained supernatant fraction is concentrated by vacuum concentration or vacuum centrifugation for approximately 5 hours while heating at 40° C. to 50° C., and then dried by freeze-drying, vacuum-heating and drying or a spray-dry method, so that the fermentation extract of the husks, pellicles and astringent skin can be obtained. The above fermentation extract can also be used as a composition for foods or medicaments. The clinical dose of the fermentation extract is 0.02 to 0.04 g/Kg body weight/day, and 0.1 g/day of the extract is normally used for an adult. The extract is preferably administered between meals, such that 0.05 g of the extract is administered in the morning and in the evening.

As Koji to be inoculated, rice koji (kome-koji), as well as oats koji (mugi-koji) or the like can be used. Further, lactic acid bacteria, yeast and the like can also be used (see below).

(1) Aspergillus: filamentous fungi belonging to the genus Aspergillus (Aspergillus oryzae)

(2) Lactic acid bacteria: those belonging to the genera Lactobacillus,
Lactococcus or Streptococcus
Lactobacillus bulgaricus
Lactobacillus delbrueckii
Lactobacillus longum
Lactobacillus acidophilus
Lactobacillus plantarum
Lactobacillus fermentum
Lactobacillus casei
Lactococcus lactis
Streptococcus lactis
Streptococcus thermophilus

(3) Yeast: those belonging to the genera Saccharomyces, Schizosaccharomyces, Kluyveromyces or Pichia
Saccharomyces cerevisiae
Saccharomyces kluyveri
Saccharomyces paradoxus
Saccharomyces pastorianus
Schizosaccharomyces pombe
Kluyveromyces lactis
Kluyveromyces marxianus
Pichia pastoris

For example, when lactic acid bacteria such as Lactobacillus bulgaricus are used, lactic acid bacteria, in an approximately 2 to 5 % by weight of the husks, pellicles and astringent skin, are used to perform lactic fermentation for 10 to 24 hours under a temperature condition of a culture temperature ranging from approximately 38° C. to 40° C., so that the lactic fermentation product of the husks, pellicles and astringent skin can be formed. Fermentation can also be performed by adding water to the husks, pellicles and astringent skin, and then steaming these materials with vapor at a normal pressure without boiling. Further, a method, which involves fermenting the husks, pellicles and astringent skin of Coix seeds together with the grains, is also performed according to the above method. When the husks, pellicles and astringent skin are fermented together with the grains, the clinical dose of the fermentation food is 0.2 to 0.4 g/Kg body weight/day, and 10 g/day of the food is normally used for an adult. The food is preferably administered between meals, such that 5 g of the food is administered in the morning and in the evening. The dose of the fermentation extract of the husks, pellicles, astringent skin and grains is 0.04 to 0.08 g/Kg body weight/day, and 2 g/day of the extract is normally used for an adult.

2. Production of the Enzyme-Treated Product of the Husks, Pellicles and Astringent Skin of Coix Seed

The enzyme-treated product of the husks, pellicles and astringent skin of Coix seed can be obtained by any existing enzyme treatment method. Specifically, Coix seeds with husks (grains with husks) are washed well with water, and then dried sufficiently. The husks of Coix seeds are then lightly ground with a rice-milling machine. After dehulling treatment, Coix seed grains are separated into undehulled grains and dehulled grains using a sieve with a mesh size of approximately 3.5 (5.6 mm). The undehulled grains are treated again with a rice-milling machine. The thus obtained husks, pellicles and astringent skin are ground using an impact grain grinder so as to be passed through a 30 mesh (0.5 mm) sieve. The ground product as a raw material is braised in the presence of various enzyme agents, so that the enzyme-treated product of the husks, pellicles and astringent skin of Coix seeds can be obtained. The enzyme-treated product is then filtered by centrifugation. The resulting supernatant fraction is concentrated, and then dried, so that the enzyme extract of the husks, pellicles and astringent skin can be obtained. Further, the above fermentation product and the fermentation extract can be subjected to further enzyme treatment.

Examples of an enzyme that can be used for this purpose include various enzyme agents such as a diastase agent, a Takadiastase agent, an α-amylase agent, a β-amylase agent, a glucoamylase agent, a pectinase agent, a β-glucosidase agent, a cellulase agent, a hemicellulase agent and a xylanase agent. Specific examples of the enzyme include KLEISTASE L-1 (produced by Daiwa kasei) as liquefying α-amylase agent, KLEISTASE T-5 (produced by Daiwa kasei) as a thermostable α-amylase agent, Sumizyme T (produced by SHIN NIHON CHEMICAL) as a saccharogenic α-amylase agent, β-amylase (produced by NAGASE & CO., LTD.) as a β-amylase agent, gluczyme (produced by AMANO ENZYME) and amyloglucosidase (produced by Novo Nordisk) as a glucoamylase agent, pectinase A (produced by AMANO ENZYME) and pectinase G (produced by AMANO ENZYME) as a pectinase agent, novozyme 188 (produced by Novo Nordisk) as β-glucosidase agent, cellulase A (produced by AMANO ENZYME) and cellulase T (produced by AMANO ENZYME) as a cellulase agent, hemicellulase “Amano” (produced by AMANO ENZYME) and cellulosin HC100 (produced by Hankyu Bioindustry) as a hemicellulase agent, and cellulosin TP25 (produced by Hankyu Bioindustry) as a xylanase agent.

The sufficient amount of the above enzyme to be added is approximately 0.1% to 5.0% by weight of the raw material. When two or more types of enzymes are used, the enzymes may be used simultaneously or used respectively at a different timing. When extraction is performed using these enzyme agents, it is suitable to use the agents by appropriately selecting enzyme concentrations and conditions. A temperature for treatment is between 45 and 85° C., and preferably between 50 and 60° C., and pH is between 3.5 and 6, and is preferably pH 5. Further, the duration for treatment is normally between 20 and 180 minutes, and preferably 90 and 120 minutes. The volume of water to be used for extraction is not specifically limited. When the extraction yield and the like are considered, the volume is preferably approximately between 3 and 7 liters per 1 Kg of Coix seeds as a raw material.

The above enzyme-treated product can be used as a composition for foods or medicaments. The clinical dose of the enzyme-treated product is 0.1 to 0.2 g/Kg body weight/day, and 5 g/day of the product is normally used for an adult. The product is preferably administered between meals, such that 2.5 g of the product is administered in the morning and in the evening. The dose of the enzyme extract is 0.02 to 0.04 g/Kg body weight/day, and 0.1 g/day of the extract is normally used for an adult. The extract is preferably administered between meals, such that 0.05 g of the extract is administered in the morning and in the evening. A method for treating with an enzyme the husks, pellicles, astringent skin and grains of Coix seeds is performed according to the above method. The clinical dose of the enzyme-treated product of the husks, pellicles, astringent skin, and grains of Coix seeds is 0.2 to 0.4 g/Kg body weight/day, and 10 g/day of the product is normally used for an adult. The product is preferably administered between meals, such that 5 g of the product is administered in the morning and in the evening. The dose of the enzyme extract is 0.04 to 0.08 g/Kg body weight/day, and 2 g/day of the extract is normally used for an adult.

The above fermentation product and enzyme-treated product may be in any form such as concentrate, liquid, paste or suspension, in addition to solid forms (powder and granule). The composition can be certainly used intact as a food or a drink. The composition can also be used, in combination with other foods or food components, as foods or drinks according to an appropriate standard method. For example, the composition as an active ingredient and working ingredients that are normally used for producing drinkable preparations can be formulated into a healthy drink or the like. Further, the composition can also be formulated into a pharmaceutical product such as tablets, capsules, granules, powder, syrup, dermatological agents and the like. These various pharmaceutical preparations can be formulated according to any standard method by using the composition as a principal ingredient and various adjuvants that are normally used in the technical field of pharmaceutical preparation, for example an excipient such as lactose and starch, a binder, a disintegrating agent, a lubricant, and a flavor.

The composition is derived from nature, and the source to be used herein has long been used, so that the composition has no toxicity or has extremely low toxicity, and thus shows great safety. Even elderly persons, babies and invalids can take the composition long term.

3. Production of the Compound of the Fermentation Product or the Enzyme-Treated Product of the Husks, Pellicles and Astringent Skin of Coix Seeds with Folic Acid

We have found that a clinical effect can be synergistically produced by compounding the fermentation product or the enzyme-treated product of the husks, pellicles and astringent skin of Coix seeds with folic acid. Combinations of compounding are as shown below.

Fermentation product+folic acid

Enzyme-treated product+folic acid

Fermentation product+enzyme-treated product+folic acid

As a folic acid product, for example, a tablet of foliamin [produced by NIHON PHARMACEUTICAL, and produced by TAKEDA CHEMICAL INDUSTRIES] (5 mg in a tablet), folic acid (10-fold diluted powder) [produced by NIHON PHARMACEUTICAL, and produced by TAKEDA CHEMICAL INDUSTRIES], or folic acid injection (1 ampule containing 15 mg of folic acid) [produced by NIHON PHARMACEUTICAL, and produced by TAKEDA CHEMICAL INDUSTRIES] can be used. The amount of folic acid to be compounded for a chemical preventive against tumors (agent for internal use) is 100 to 800 μg per day, and preferably 400 μg.

When folic acid is compounded into a remedy for internal use against precancerous lesions including cervix dysplasia and laryngeal papillomatosis, early carcinoma including cervical carcinoma (stage 0), condyloma acuminatum, verruca vulgaris, adolescent verruca plana and senile verruca, relatively a large amount of folic acid is administered. For example in a preferable method, the dose is gradually decreased from 15 mg/day of folic acid in the first 4 weeks, 10 mg/day in the following 2 weeks, and then 300 μg to 1 mg/day in the following days.

As a preventive or a remedy (agent for internal use) against molluscum contagiosum, follicular keratosis, chloasma, freckle, wrinkle, senile plaque, skin pigmentation, skin roughness, clavus, acne vulgaris (acne), constipation or the smell of stool, 100 to 800 μg/day, and preferably 400 μg/day of folic acid is compounded. When a folic acid injection is used, the above dose of the injection can be administered subcutaneously, intramuscularly, or intravenously.

When folic acid is compounded, various pharmaceutically acceptable carriers can be compounded. Specifically, excipients including natural and synthetic macromolecules such as cellulose and a derivative thereof and starch and a derivative thereof, stearic acid and a salt thereof, lubricants such as natural and synthetic wax, saccharides, sour agents, perfume and the like can be compounded. Various dosage forms (agents for oral administration and the like) that can be selected according to administration methods and routes of administration include powders, granules, tablets, pills, hard capsules, soft capsules, syrups, drinks and the like. As an agent for oral administration, a powder is preferred. Lactose, starch or the like is particularly preferable for use as an excipient to be compounded. In addition to folic acid, other vitamins, for example, vitamin B12, B1, B2, B3 and B6, vitamin C, vitamin E or vitamin A can be added, or a nucleic acid can be compounded. When a nucleic acid is compounded, for example, 1000 mg/day of a salmon milt extract is used. When a large amount of folic acid is administered over several months, 5 to 15 mg/day, and desirably 10 mg/day of zinc is compounded to take care of zinc deficiency. Further, the composition of the present invention may be used not only as a medicament, but also as a functional food.

4. Compound Containing the Fermentation Product or the Enzyme-Treated Product of the Husks, Pellicles and Astringent Skin of Coix Seed and Denshichi Ginseng

We have found that the clinical effect can be greatly enhanced by further compounding Denshichi ginseng into the fermentation product or the enzyme-treated product of the husks, pellicles and astringent skin of Coix seed or the same compounded with folic acid. Combinations of compounding are as shown below.

Fermentation product+Denshichi ginseng

Enzyme-treated product+Denshichi ginseng

Fermentation product+enzyme-treated product+Denshichi ginseng

Fermentation product+Denshichi ginseng+folic acid

Enzyme-treated product+Denshichi ginseng+folic acid

Fermentation product+enzyme-treated product+Denshichi ginseng+folic acid

Denshichi ginseng that can be used in the present invention may be any natural product, normally cultivated product or tissue culture product. Further, ginseng in the form of dry bulk powder, hot water extract, alcohol extract, fermented Denshichi ginseng extract, enzyme-treated Denshichi ginseng and the like can be used. Preferably, Denshichi ginseng to be used herein is prepared by lightly immersing Denshichi ginseng in hot water at 100° C. so as to suppress germination, drying the ginseng, and then grounding into impalpable powders (Denshichi ginseng bulk powders). Manufactured forms of the powder to be used herein are, for example, Densanshichi powders [produced by TOCHIMOTO TENKAIDO].

In the present invention, the amount of Denshichi ginseng bulk powders to be compounded differs depending on age, symptom and the like, and is between 1 and 12 g, and preferably 6 g per day. The number of oral administration can be once a day or several different times a day. When Denshichi ginseng is compounded, various pharmaceutically acceptable carriers can be compounded. Specifically, excipients including natural and synthetic macromolecules such as cellulose and a derivative thereof, starch and a derivative thereof, stearic acid and a salt thereof, lubricants such as natural and synthetic wax, saccharides, sour agents, perfume and the like can be compounded thereinto. Examples of various dosage forms for oral administration that can be employed according to administration methods and routes of administration include powders, granules, tablets, pills, hard capsules, soft capsules, syrup, drinks and the like. As an agent for oral administration, the powder form is preferred. It is particularly preferable to use lactose, starch or the like as an excipient to be compounded thereinto. The agent of the present invention may be used not only as a medicament, but also as a functional food.

5. Isolation and Confirmation of the Activity of a Novel Effective Substance in the Fermentation Product or the Enzyme-Treated Product of Coix Seed

Recently, based on a new concept wherein a compound showing a cancer-preventive effect is extracted from a substance recognized to have the effect, the amount of this compound is increased, and the increased compound is thus returned to the original substance so as to further enhance the effect, so-called Designer's Foods or Designer's Drugs have been produced. The side effect is low as a matter of course, so that they are actually sold as cereal foods such as bread and cookies. Also in the present invention, effective substances in the fermentation product or the enzyme-treated product of Coix seed can be used for the Designer's Foods or Designer's Drugs. In the present invention, an oligosaccharide fraction (described later) can also be used alone or in combination by compounding with the fermentation product or the enzyme-treated product. The dose of C3 fraction is 0.5 to 2 g/day, and preferably 1 g/day, and C4 fraction 0.1 g to 1 g/day, and preferably 0.5 g/day.

6. Remedy or Preventive

The composition of the present invention can be used as remedies or preventives against various diseases. Examples of diseases include the following diseases. The composition of the present invention can be used for any case such that any one of the following diseases is developed alone, two or more of the diseases are developed at the same time, or one disease is complicated with other diseases. (1) Tumors

At least one type of tumor selected from the group consisting of benign tumor, precancerous lesion, uterine cancer, vaginal cancer, vulvar cancer, skin cancer, esophageal cancer, oral cavity cancer, gingival cancer, jaw cancer, pharyngeal cancer, vocal cord carcinoma, lung cancer, bladder cancer, thyroid cancer, breast cancer, gastric cancer, colon cancer, pancreatic cancer, renal cancer, ovarian cancer, melanoma, tumor of central nervous system, tumor of peripheral nervous system, mediastinal tumor, hepatic cancer, bile duct cancer, gallbladder cancer, tumor of renal pelvis, ureter cancer, testicular tumor, prostate cancer, choriocarcinoma, fallopian tube cancer, sarcoma, leukemia, erythroleukemia, multiple myeloma, malignant lymphoma and carcinosarcoma.

In the above tumors, a benign tumor includes soft part tumor, polyp of colon and vocal cord polyp, of which soft part tumor includes a giant cell tumor of the tendon sheath. Further, the precancerous lesion includes cervical dysplasia and tylosis linguae.

(2) Human Papillomaviral diseases

At least one type of disease selected from the group consisting of condyloma acuminatum, verruca vulgaris, adolescent verruca plana, senile verruca and laryngeal papillomatosis.

(3) At least one type of condition selected from the group consisting of molluscum contagiosum, follicular keratosis, chloasma, freckle, wrinkle, senile plaque, skin pigmentation, skin roughness, clavus and acne vulgaris.

(4) Constipation or the smell of stool

### BRIEF DESCRIPTION OF DRAWINGS

FIG. 1 is colposcopic images of the uterine cervix after application with acetic acid.

### BEST MODE FOR CARRYING OUT THE INVENTION

The present invention will be further described by the examples. However, the technical scope of the present invention is not limited by these examples.

### EXAMPLE

### Production of the Fermentation Product of the Husks, Pellicles and Astringent Skin of Coix Seed

Coix seeds with husks (grains with husks) were washed well with water, and then dried sufficiently. The husks of Coix seeds were then lightly ground with a rice-milling machine. Coix seed grains were separated into undehulled grains and dehulled grains using a sieve with a mesh size of approximately 3.5 (5.6 mm). Thus the raw material of the husks, pellicles and astringent skin was obtained. 5 liters of water were added to 1 kg of husks, pellicles and astringent skin, so that these materials were immersed in water for 1 hour. The solution was gradually heated for 30 minutes to be boiled, followed by further boiling of 30 minutes. Subsequently, the solution was concentrated by vacuum centrifugation for 5 hours while being heated to 40° C. to 50° C. 200 g of rice koji, which had been prepared by allowing Aspergillus (Aspergillus oryzae) to directly grow on commercial steamed rice, was added to the 1 Kg of the raw material of husks, pellicles and astringent skin. Fermentation was performed for 48 hours at 30° C. while agitating the mixture, followed by sterilization at around 90° C. for approximately 30 minutes. The sterilized product was cooled and then dried by a spray-dry method, so that the fermentation product of the husks, pellicles and astringent skin of Coix seeds was obtained. Further, the product subjected to 48 hours of fermentation was sterilized at around 90° C. for approximately 30 minutes, and then filtered by centrifugation, thereby obtaining a supernatant fraction. The fraction was concentrated by vacuum centrifugation for 5 hours while being heated at 40° C. to 50° C., and then dried by the spray-dry method, thereby obtaining the fermentation extract of the husks, pellicles and astringent skin.

### EXAMPLE 2

### Production of the Enzyme-Treated Product of the Husks, Pellicles and Astringent Skin of Coix

One kg of the husks, pellicles and astringent skin of Coix which had been ground so as to be passed through 30 mesh (0.5 mm) was added with five liter of water and soaked in water for 6 hours. 10 g of hemicellulase (produced by AMANO ENZYME: hemicellulase “AMANO”) and 10 g of pectinase (produced by AMANO ENZYME: pectinase G “AMANO”) were respectively mixed thereinto, followed by an enzyme reaction at 45° C. for 120 minutes. Subsequently, the mixture was gradually heated and maintained at 75 to 80° C. for approximately 40 minutes. The mixture was then further heated and gently boiled for 30 minutes. After concentrated by vacuum centrifugation for 5 hours while being heated at 40° C. to 50° C., the mixture was dried by the spray-dry method. Thus the enzyme-treated product of the husks, pellicles and astringent skin was produced.

Further the supernatant fraction, which had been obtained by filtering the braised material by centrifugation, was concentrated by vacuum centrifugation for 5 hours while being heated at 40° C. to 50° C. Then, the product was dried by the spray-dray method, so that the enzyme extract of the husks, pellicles and astringent skin was produced. By this method, approximately 200 g of the extract per 1 Kg of husks, pellicles and astringent skin was collected.

### EXAMPLE 3

### Isolation of Active Fraction

(1) Fractionation of the Enzyme Extract of Coix Seed

The enzyme extract of Coix seeds with husks was dialyzed using Spectra/Por 1 (50×31, 8 mm×30 m) as a dialysis membrane. 2 or 3 drops of acetic acid were added to a 500 mL bottle. The dialysis membranes cut to have an approximate length of 30 cm were then immersed in the bottle for 5 minutes. This step is to form a chelate with chalybeate contained in the dialysis membrane so as to remove chalybeate. Next, the dialysis membrane was immersed in distilled water for 5 minutes, and then washed with distilled water.

After 15 g of the sample enzyme extract of Coix seeds with husks was dissolved in 500 mL of distilled water, the prepared sample was put into the dialysis membrane with its end tied to approximately one third in height of the membrane. At this time, air was completely drawn out. The other end of the dialysis membrane was tied, and then the membrane was put into a 5 L Erlenmeyer flask. Dialysis was performed against 2.5 to 3.0 liters of distilled water. The external solution of the dialysis membrane was concentrated using an evaporator, freeze-dried, and then weighed. Approximately 3 liters of distilled water was added again to the internal solution. The solution was then agitated, followed by dialysis for 3 days. This step was repeated 5 times. The weights of the external solution measured 5 times were added up. The final remaining external solution was concentrated, freeze-dried, and then weighed. The thus obtained dialyzed internal solution was a high molecular fraction and the external solution was a low molecular fraction. Respectively obtained proportions were also calculated.

(2) Dialysis and Fractionation of the Enzyme Extract of Coix Seed

26.0 g of the enzyme extract of Coix seed was dialyzed using a dialysis membrane (molecular weight cutoff between 6000 and 8000), and then concentrated using an evaporator, thereby obtaining 1.5 g (5.8%) of a high molecular fraction (internal solution) and 21.4 g (82.3%) of a low molecular fraction (external solution).

Among 21.4 g of the low molecular fraction, 13.0 g of the same was fractionated by silica gel chromatography. BAW (n-butanol:acetic acid:water) was used as a solvent for elution. Depending on mixture ratios, BAWs were classified into BAW-1 (6:1:2, respectively), BAW-2 (5:1:2, respectively) and BAW-3 (4:1:2, respectively). 4 liters of each of these BAWs was made to flow in sequence.

As a result, C-1 fraction (1.19 g, 7.5%) and C-2 fraction (0.32 g, 2.0%) were obtained by elution with the mixed solvent BAW-1, C-3 fraction (5.10 g, 38.6%) was obtained by elution with BAW-2, and C-4 fraction (1.57 g, 9.9%) was obtained by elution with BAW-3.

These fractions were subjected to silica gel chromatography (chloroform-methanol, 3:1 elution) to isolate a principal ingredient. The principal ingredient was subjected to instrumental analysis including mass spectroscopy, nuclear magnetic resonance spectra and the like, revealing that the C-2 fraction mainly contained glucose, and C-3 and C-4 fractions mainly contained oligosaccharides.

That is, from C-2, the obtained glucose yield was 0.26% of the enzyme extract of Coix seeds.

[MS m/z 181 (M)⁺]

From C-3, maltose was obtained at the yield of 8.6% of the enzyme extract of Coix seeds.

[MS: m/z 365 (M+Na)⁺, 343 (M+H)⁺, ¹H NMR (ppm): 3.22 (t, J=8.5 Hz), 3.36 (d, J=9.5), 3.5-3.9 (m), 4.59 (d, J=7.8), 5.17 (br s), 5.35 (br s).]

From C-4, maltotriose was obtained at the yield of 4.4% and the maltotetraose was obtained at the yield of 3.9% of the enzyme extract of Coix seeds.

Maltotriose [MS: m/z 527 (M+Na)⁺, 505 (M+H)⁺, ¹H NMR (ppm): 3.22 (t, J=8.5 Hz), 3.36 (d, J=9.5), 3.5-3.9 (m), 4.64 (d, J=8.1), 5.17 (d, J=3.9), 5.33 (d, J=3.4).]

Maltotetraose [MS: m/z 689 (M+Na)⁺, 667 (M+H)⁺, ¹H NMR (ppm): 3.23 (t, J=8.5 Hz), 3.38 (d, J=9.5), 3.5-4.0 (m), 4.61 (d, J=8.3), 5.19 (br s), 5.35 (br s).]

Among these fractions, C-1, C-3 and C-4 were orally administered to mice so as to examine antineoplastic activity.

(3) Antineoplastic Activity Test (in vivo) of each Fraction of the Enzyme Extract of Coix Seeds

Cancer cells (Sarcoma-180) were administered to mice intraperitoneally, and then subcultured for 1 week. The mouse ascites was then diluted to 200-fold by adding physiological saline to 201 μl of the ascites. Subsequently, the concentration of cancer cells was prepared to be 0.4 to 1.0×10⁶ cells/mL.

0.05 mL of the solution of cancer cells prepared as described above was transplanted subcutaneously using a syringe into the base of the right extremity of a mouse. On a day after transplantation, the mice were randomly divided into cages, so that total body weights of mice of these cages became uniform. Grouping was performed so that each cage contained 6 mice.

In the meantime, samples of each fraction of the enzyme extract of Coix seeds were diluted with distilled water to be 10 mg/mL. Distilled water was used as a control. 10 days after transplantation, 0.01 mg/g (body weight) of the thus prepared samples of each fraction was orally administered using a probe.

To measure antineoplastic activity, at first mouse body weight was measured everyday during administration of the samples after transplantation, and then measured at an interval of once every 3 days to examine the side effect of the drug. Tumor size was measured at an interval of once every 3 days during 1 to 5 weeks after transplantation. In this method, a diameter and a short diameter were measured in centimeters, and then the values are used to calculate (diameter×short diameter²)/2W, thereby obtaining provisional measurements for tumor volumes. The mouse tumors were excised using ophthalmologic surgical scissors, and then weighed. The average values of each cage were calculated for each measured mouse body weight, tumor volume and tumor weight, and then the activity was calculated.

The activity was calculated based on the control.
Activity=100×{tumor weight (control)−tumor weight (sample)}/tumor weight (control)

As a result, among the fractions containing oligosaccharides, cancer cell growth-inhibiting activity was observed in the C-3 fraction and the C-4 fraction (C-3 fraction: 79%, C-4 fraction: 44%), of which particularly strong activity was observed in the C-3 fraction. In the above fractions, any side effects such as anorexia, body weight loss and the like were not found in the mice.

### EXAMPLE 4

### Cervical Carcinoma in situ (CIS) (the International Classification of Cervical Cancer: stage 0)

Method:

A method for the clinical testing was performed in the manner described below under sufficient informed consent. Colposcopy was performed for patients who had been diagnosed to be class IV or V based on the cytobiosis of cervical cancer, portions of their lesions were subjected to biopsy, and then administration of various pharmaceutical preparations was immediately started. Test groups are as shown below. Each group was observed.

Control group (42 cases): a non-treated group

Group A (21 cases): commercial hot water extract of Yokuinin (daily dose of 2 g of the extract ingredient)-administered group

Group B (15 cases): hot water extract of Coix seeds with husks (8 cases)-administered group, or ethanol extract of Coix seeds with husks (7 cases)-administered group (wherein each received daily doses of 2 g of the respective extract ingredients)

Group C (8 cases): folic acid (15 mg/day)-administrated group

Group D (12 cases): enzyme extract of Coix seeds with husks (6 cases)-administered group or fermentation extract (6 cases)-administered group (wherein each received daily doses of 2 g of the respective extract ingredients)

Group E (11 cases): the enzyme extract of Coix seeds with husks compounded with folic acid (15 mg/day) (5 cases)-administered group or fermentation extract compounded with folic acid (15 mg/day) (6 cases)-administered group (wherein each received daily doses of 2 g of the respective extract ingredients)

1 to 2 weeks after administration, the biopsy results were obtained. Administration of the pharmaceutical preparation was continued only for patients who had been shown to be stage 0 (CIS) based on the International Classification of cervical cancer. The other patients were considered as dropout cases, and the administration of the pharmaceutical preparation to these patients was discontinued. In addition, administration of the pharmaceutical preparation was continued until one day before the implementation of ectocervical conization or panhysterectomy. Only patients who could have continuously taken the pharmaceutical preparation for at least 2 weeks or longer were subjected to the study. Administration of the pharmaceutical preparations was discontinued at week 6 at the longest. Subjects of the control group (non-treated group) were randomly extracted from those who had never been administered with foods nor drugs according to the past clinical history.

Results:

None of the cases of the control group (42 cases) showed the disappearance of carcinoma in situ as a result of postoperative histological tests. Further none of the cases of groups A, B and C showed the disappearance of cancer. However, 5 cases of group D showed the disappearance of cancer (there was a significant difference with p<0.001 compared with the control group), and 7 cases of group E showed the same (there was a significant difference with p<0.001 compared with the control group).

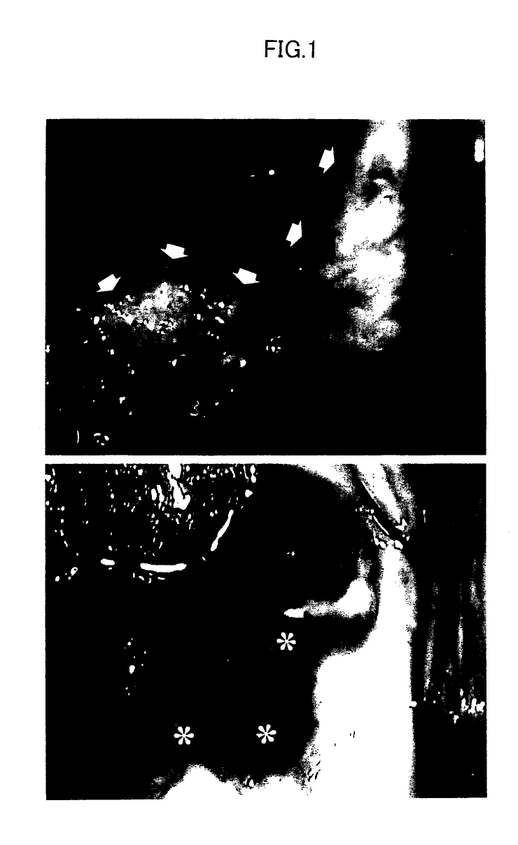

One of the above 7 cases (group E) wherein a complete response has been achieved is shown below (FIG. 1).

A 53 years old patient, diagnosed to be class V based on cytological diagnosis in cancer screening.

A photograph on the top shows white epithelium after application with acetic acid in the direction between 3 o'clock and 6 o'clock of uterine cervix at the time of visit. White epithelium was observed all over the uterine cervix. Biopsy was performed in the 8 o'clock direction, so that the patient was diagnosed as CIS. Administration of the enzyme extract of Coix seeds with husks (2 g/day of the extract)+15 mg/day of folic acid was started at the time of visit with informed consent.

The lower panel in FIG. 1 is an image that was taken after application with acetic acid in the direction between 1 o'clock and 5 o'clock on day 21 after administration. White epithelium disappeared completely.

On day 35 after administration, conization of the cervix of the uterus was performed, followed by close pathological investigation. All the cancers disappeared.

In addition, arrows in FIG. 1 indicate white epithelium and the symbol “*” indicates the positions of lesions after cure.

### EXAMPLE 5

### Cervical Carcinoma in situ (CIS) (International Classification of Cervical Cancer: stage 0)

The case presented below is that a complete response was achieved against carcinoma in situ during pregnancy by a method in which the enzyme extract of Coix seeds with husks is compounded with folic acid.

(1) Case: a 29 Years Old Primigravida

The patient was diagnosed to be class V based on cytological diagnosis of uterine cancer during a periodic medical examination on week 7 of pregnancy. White epithelium was observed all over the uterine cervix after application with acetic acid in colposcopy on week 9 of pregnancy. A part of the white epithelium was subjected to biopsy, so that it was diagnosed as carcinoma in situ. Therapy including conization was explained, but the patient refused the therapy in fear of an abortion risk. With sufficient informed consent, the enzyme extract of Coix seeds with husks (daily dose of 2 g of the extract ingredient) was administered from week 12 of pregnancy for 20 days. However, no vanishing tendency of the white epithelium was observed. Hence, the enzyme extract of Coix seeds with husks compounded with folic acid (2 g/day of the extract) was used instead. In view of pregnancy, the amount of folic acid compounded in this case was gradually decreased from 15 mg/day for 2 weeks, followed by 10 mg/day for 1 week, 5 mg/day for 1 week and to 400 μg/day for 1 week. According to the findings as observed by a colposcope on week 1 after administration of the enzyme extract of Coix seeds with husks compounded with folic acid, white epithelium was still present and no change was found. However, on week 2, even after application with acetic acid, the white epithelium developed light pink color tone and a vanishing tendency was observed. When observation was further continued, lesions disappeared on week 4 after administration. Consequently, administration of the enzyme extract of Coix seeds with husks was temporarily discontinued for 6 weeks in total. Then, to prevent recurrence, the enzyme extract of Coix seeds with husks (2 g/day of the extract)+folic acid (400 μg/day) was administered for 3 weeks from week 31 of pregnancy. Further cytological diagnose on weeks 21, 31 and 37 of pregnancy showed class II (normal). Attention was paid on the side effects during administration of the Coix seed extract. As a result, no tendencies of abortion nor early delivery were found, and the patient delivered a 3250 g normal baby girl at week 40 of pregnancy. Since the result of cytological diagnosis on 1 month after delivery was class II, and both cytological diagnosis and colposcopy on 2 months after delivery showed no abnormalities, follow-up was performed without conization. Because of the request from the patient herself, a pharmaceutical preparation containing the enzyme extract of Coix seeds with husks (2 g/day of the extract) compounded with folic acid (400 μg/day) was administered for only 1 to 2 months a year. 3 years after the delivery of the first child, the patient delivered another baby. Now, 9 years have passed since the first visit, and no abnormality was found by various tests including cytological diagnosis.

(2) Case Summary

This is an important case that has provided us an occasion to discover that the addition of folic acid to the enzyme extract synergistically improves its clinical effect. According to our experience, administration of the enzyme extract of Coix seeds with husks alone to carcinoma in situ during pregnancy is wasteful, which is similar to condyloma acuminatum accompanying pregnancy. However, compounding with folic acid leads to the advent of a synergistic effect.

### EXAMPLE 6

### Cervical Dysplasia

According to the CIN classification, CIN1 and CIN2 correspond to mild dysplasia and moderate dysplasia, respectively, and CIN3 includes severe dysplasia and the above CIS. Here, the clinical effect of the pharmaceutical preparations of the present invention against the CIN1 and CIN2 is presented.

A test was conducted for cases, which had been diagnosed to be class III by cytological diagnosis and as having mild dysplasia or moderate dysplasia by histological diagnosis, and carefully followed up without conization, and for which informed consent had been obtained.

(1) Methods and Results

Among cases diagnosed as CIN1 or CIN2, none of the cases of the group for which no pharmaceutical preparation had been administered (15 cases) showed improvement in both cytological diagnosis and colposcopy findings 2 months after diagnosis. In the commercial hot water extract of Yokunin (8 cases)-, the hot water extract of Coix seeds with husks (3 cases)- or the ethanol extract of Coix seeds with husks (3 cases)-administered group (daily dose of 2 g of the respective extract ingredients) (14 cases), no case showed improvement in both cytological diagnosis and colposcopy findings 2 months after administration. None of the cases of the folic acid (15 mg/day)-administered group (5 cases) showed improvement under similar conditions. Further, the enzyme extract of Coix seeds with husks (3 cases)- or the fermentation extract (3 cases) (daily dose of 2 g of the respective extract ingredients)-administered group (6 cases), the enzyme extract of Coix seeds with husks (4 cases) or the fermentation extract (4 cases) (respective daily dose of 2 g as the extract ingredient) compounded with folic acid (15 mg/day)-administered group (8 cases) were observed. Only 1 case for each group showed improvement in both cytological diagnosis and colposcopy findings 2 months after administration. Hence, long-term administration of the food was attempted. Test groups are as shown below. Each group was observed.

Control group (71 cases): non-treated group

Group A (24 cases): commercial hot water extract of Yokuinin (10 cases), hot water extract of Coix seeds with husks (7 cases) or ethanol extract of Coix seeds with husks (7 cases) (daily dose of 2 g of the respective extract ingredients)-administered group

Group B (25 cases): enzyme extract (10 cases) or fermentation extract (15 cases) of Coix seeds with husks (daily dose of 2 g of the respective extract ingredients) compounded with folic acid (400 μg/day)-administered group

Each pharmaceutical preparation was administered over a long period (1 year), and cytological diagnosis and colposcopy were performed every 3 to 4 months. When no abnormality was found by cytological diagnosis, it was defined as a clinical cure. As a result of cytological diagnosis, 12 cases out of the control group (71 cases) showed improvement to class II or less, 5 cases out of group A (24 cases) showed improvement (no significant difference with p>0.05 compared with the control group), and 15 cases out of group B (25 cases) showed improvement (there were significant differences with p<0.001 and p<0.01 compared with the control group and group A, respectively).

(2) Case Summary

The results showed that although cervical dysplasia CIN1 and CIN2 are relatively mild lesions which may shift to carcinoma in situ in the future, carcinoma in situ is more easily cured. Aside from the explanation of this paradox, the method of the present invention against CIN1 and CIN2 is superior to the method using the hot water extract of Yokuinin, or the hot water extract/ethanol extract of Coix seeds with husks. Moreover, we have recently confirmed cases wherein CIN1 and CIN2 were cured earlier by the use of a combination of the enzyme extract or fermentation extract of Coix seeds with husks (2 g/day of the extract), folic acid (400 μg/day) and Denshichi ginseng.

### EXAMPLE 7

### Advanced Cancer

(1) Case: 76 Years Old, Esophageal Cancer, (Squamous Cell Carcinoma), Stage IVB

A terminal cancer patient, who was diagnosed at the first visit as having progressive esophageal cancer at stage IVB (with multiple liver metastasis), conservatively followed up, and had received esophageal dilation every week. The enzyme extract of Coix seeds with husks (2 g/day of the extract) was administered to the patient with first-person informed consent. From 2 weeks after the administration, the patient became able to smoothly ingest without esophageal dilation. The QOL (quality of life) of the patient was improved.

(2) Case Summary

This case shows the effect of the enzyme extract of Coix seeds with husks against esophageal cancer. The patient was very pleased that the patient became able to eat foods.

### Example 8

### Benign Tumor

(1) Case of Disappearance of a Benign Tumor, Giant Cell Tumor of Tendon Sheath

Case: 56 years old, male

The patient discovered a tumor with a diameter of 8 mm on the back of his left foot 2 years ago, and had it biopsied by a local doctor. He was diagnosed as having giant cell tumor of tendon sheath and was recommended to receive an operation, but received no treatment because of its light symptoms. Recently he found an increase in the tumor, and visited the hospital worrying about the tumor. When the enzyme extract of Coix seeds with husks (2 g/day of the extract) was administered with first-person informed consent, the tumor disappeared on week 7. Subsequently, the administration of 1 g/day of the extract was continued, no relapse was observed.

Case Summary

Pathological diagnosis for this case has been confirmed, suggesting that this is a case of cure with high reliability. There have been very few reports that the benign soft part tumor disappeared by the use of some type of drugs. This was diagnosed as a case wherein a complete response was achieved by the enzyme extract of Coix seeds with husks.

(2) Case of Disappearance of a Benign Tumor, Vocal Cord Polyp

Case: 53 years old, female

Symptoms such as husky voice, dysphonia, unable to raise her voice and laryngeal foreign-body sensation appeared a year ago. The patient was diagnosed as having vocal cord polyp (with a diameter of approximately 1 cm) by a local otolaryngologist. No tendency of improvement in the symptoms was found after 2 months of conservative follow-up, so that the patient was recommended to have an operation by the doctor. However, the patient received no treatment. This time, the patient found the exacerbation of the symptoms and visited the hospital. Vocal cord polyp, which was the same size and located at the same position as of the one found a year ago, was found by examination, so that the administration of a compounding agent of the enzyme extract of Coix seeds with husks (2 g/day) and Denshichi ginseng (8 g/day) with informed consent was started. Since the patient became able to have her voice smoothly on day 4 after the start of administration, the agent was changed to a compounding agent of the enzyme extract of Coix seeds with husks (2 g/day), Denshichi ginseng (8 g/day) and folic acid (15 mg/day). On day 20 after the start of the therapy, all the symptoms disappeared. When the patient visited the otolaryngologist, the vocal cord polyp had completely disappeared.

Case Summary

This is a case wherein the vocal cord polyp that had existed for approximately a year disappeared after 20 days of the therapy. The patient was diagnosed by a medical specialist. This case was diagnosed as a case wherein a complete response was achieved by the compounding agent containing the above 3 types of ingredients.

### EXAMPLE 9

### Condyloma Acuminatum (Human Papillomaviral Disease)

Methods and Results

Subjected 8 cases were of 18 to 28 years old patients with condyloma acuminatum of vulva who had taken the hot water extract of Yokuinin (daily dose of 2 g of the extract ingredient) for at least 4 weeks and the therapy was determined as invalid. After a dose-free period of at least 2 weeks had been provided, the enzyme extract of Coix seeds with husks (2 g/day of the extract) was administered for 4 weeks to 6 weeks to the 8 cases with informed consent. As a result, 4 cases showed the disappearance of condyloma acuminatum (cases of complete response), 1 case showed an improvement (a case of partial response), and 3 cases showed no effect. All the 3 cases showing no effect were of pregnant women complicated with condyloma acuminatum.

### EXAMPLE 10

### Condyloma Acuminatum (Intractable Case)

A case of a pregnant woman wherein a successful result was obtained by compounding with folic acid is as presented below.

(1) Case: Diagnosis: Week 15 of Pregnancy, Condyloma Acuminatum of Vulva, Vagina and Vaginal Portion of Cervix

The patient had felt discomfort and itching at the vulva from around week 12 of pregnancy. She visited our clinic at week 15 of pregnancy, and diagnosed as having condyloma acuminatum of vulva, vagina and vaginal portion of cervix (severe case). The hot water extract of Yokuinin (daily dose of 2 g of the extract ingredient) was administered with sufficient informed consent for 4 weeks, but the therapy was totally invalid. The way of administration was changed to 2 weeks of administration of the enzyme extract of Coix seeds with husks (2 g/day of extract). The outgrowth of condyloma was suspended, but no tendency of disappearance was observed. This case was diagnosed as intractable condyloma acuminatum, and the enzyme extract of Coix seeds with husks compounded with folic acid (2 g/day of the extract) was administered. The amount of folic acid compounded was gradually decreased from 15 mg/day for 1 week, 10 mg/day for 1 week, and then to 5 mg/day for 1 week. When visually inspected, no apparent change was observed in the condition at 1 week after administration. However, it was found that condyloma could be easily removed when it was picked up with forceps. The lesions of the condyloma were reduced in half on week 2, and then all the lesions disappeared on week 3. Subsequently, the dose of the enzyme extract of Coix seeds with husks was reduced to 1 g/day and that of folic acid to 400 μg/day. This administration was performed for 2 weeks, and then discontinued. During this administration period, no signs of abortion or early delivery was observed at all. The patient vaginally delivered a baby boy at week 39 of pregnancy, and no infection of condyloma acuminatum was found in the baby.

(2) Case Summary

This is a case which led us to believe that when the enzyme extract is compounded with folic acid, the enzyme extract significantly improves the clinical effect. Most cases of condyloma acuminatum accompanying pregnancy are intractable, and the affected area often significantly spreads not only over the vulva, but also over the vagina and the vaginal portion of cervix as observed in this case. Thus, other therapies such as a freezing and coagulating method cannot be applied to the cases, so that they become impossible to be treated in most cases. Most cases of condyloma acuminatum accompanying pregnancy go into remission after the completion of pregnancy. However, to effect a cure during pregnancy still makes sense, because vaginal delivery with uncured condyloma increases the amount of bleeding upon delivery, and the skin or the airway of the baby becomes infected with condyloma. It is said that condyloma often exacerbates because of lowered immunological functions during pregnancy. Therefore, this method can be considered as an extremely useful therapy. Here, attention should be paid on a folklore that the use of Coix seeds during pregnancy results in abortion or early delivery. However, there is no report that abortion or early delivery has been actually caused, and we have encountered no such case. Accordingly, it is considered that administration of the enzyme extract of Coix seeds with husks compounded with folic acid can be an extremely effective method even during pregnancy if conducted under a doctor's supervision.

### EXAMPLE 11

### Verruca Vulgaris, Adolescent Verruca Plana, Senile Verruca (Human Papillomaviral Disease)

(1) Methods and Results

The enzyme extract of Coix seeds with husks (2 g/day of the extract) and folic acid were administered simultaneously to 9 cases with verruca vulgaris, 5 cases with adolescent verruca plana and 8 cases with senile verruca for which the hot water extract of Yokuinin had been diagnosed as having no effect. The amount of folic acid administered was gradually decreased from 15 mg/day for 1 week, 10 mg/day for 1 week, and then 5 mg/day for 1 week followed by 400 μg. The effect was evaluated 3 months later. The results were: among 9 cases with verruca vulgaris, 4 cases of disappearance (complete response), 1 case of remission (partial response) and 4 cases of treatment failure. Among 5 cases with adolescent verruca plana, the results were: 1 case of disappearance (complete response), 2 cases of remission (partial response) and 2 cases of treatment failure. Among 8 cases with senile verruca, the results were 1 case of disappearance (complete response), 2 cases of remission (partial response) and 5 cases of treatment failure.

(2) Case Summary

The enzyme extract of Coix seeds with husks was shown to have an effect against in order of verruca vulgaris, adolescent verruca plana and senile verruca. There were cases with senile verruca that were difficult to cure with the use of the extract. However, since all the cases were resistant to the existing Yokuinin, the enzyme extract of Coix seeds with husks is considered to be usable.

### EXAMPLE 12

### Laryngeal papillomatosis (Human Papillomaviral Disease)

(1) Case: 75 Years Old Male

The patient received right pneumonectomy because of lung cancer 2 year ago. In this time, the patient visited complaining of hoarse voice. At 3 positions of the glottis, laryngeal papillomas, specifically, two 4-mm lesions and one 6-mm lesion, were found. This was diagnosed as multiple laryngeal papillomatosis, and ablative operation was planned two weeks away. From day 3 after the diagnosis, in addition to the enzyme extract of Coix seeds with husks (2 g/day of the extract)+folic acid (15 mg/day), administration of vitamin B1 (75 mg/day)+vitamin B6 (75 mg/day)+vitamin B12 (750 μg/day)+vitamin E (150 mg/day) was started. 10 days later, the two 4-mm lesions of laryngeal papillomas disappeared, the 6-mm lesion was decreased in size to a 3-mm lesion, and hoarseness was also significantly improved. When the administration was continued for another week, both the 3-mm papilloma and horaseness disappeared. Since then, the enzyme extract of Coix seeds with husks (2 g/day of the extract)+folic acid (1 mg/day) and vitamin B1 (50 mg/day)+vitamin B6 (50 mg/day)+vitamin B12 (500 μg/day)+vitamin E (100 mg/day) have been administered together, and no relapse of laryngeal papillomatosis has been observed.

(2) Case Summary

Papilloma is a benign tumor that develops from the epithelium into a papilliform, and occurs most frequently among benign tumors of the larynx. This is thought to be caused by infection with human papillomaviruses. It often develops at the glottis, specifically the upper portion of the glottis. If the papilloma is multiple papillomatosis as in this case, it is normally intractable. When papillomatosis is monostotic, it can relatively be easily treated. Hoarseness is the most frequently-developed symptoms. When a papilloma grows larger, it causes dyspnea. This is considered as a case wherein hoarseness was improved as the tumors disappeared, and the enzyme extract of Coix seeds with husks+folic acid exerted the effect. As in this case, we have shown that the effect can be enhanced by a combined use of other vitamin preparations such as vitamin B1, vitamin B6, vitamin B12, vitamin E, vitamin A and the like. Papillomas may start to disappear only after 10 days of administration as presented in this case. Normally, a complete cure often takes at least 3 weeks. Long-term administration of interferon is employed against laryngeal papillomatosis, but it is thought to be difficult to cure the disease completely by this way. Further, when a lesion spreads to cause dyspnea, complete removal of the larynx may be performed. Hence, the noninvasive enzyme extract of Coix seeds with husks can be said to be a pharmaceutical composition having extremely high usability.

### EXAMPLE 13

### Molluscum Contagiosum

(1) Case: 3 Years Old Boy

The patient visited the hospital, because 20 to 30 molluscum contagiosum lesions were found from the trunk to the axillary region 3 months ago. A Yokuinin extract (2 g/day) was orally administered from the day of visit, and the patient was followed up for 1 week. Since almost no change was observed, the enzyme extract (2 g/day) of the husks, pellicles, astringent skin, and grains of Coix seeds was administered with the parents' informed consent. Because the number of papulae started to gradually decrease around 1 week after the administration, a compounding agent of 2 g of the enzyme extract of the husks, pellicles, astringent skin and grains, 200 μg of folic acid and 1 g of Denshichi ginseng was administered per day for 2 weeks from week 2. Thus, all the papulae of molluscum contagiosum disappeared.

(2) Case Summary

This is considered to be a case wherein a complete response was achieved by the compounding agent of the enzyme extract of the husks, pellicles, astringent skin and grains, folic acid and the Denshichi ginseng.

### EXAMPLE 14

### Keratosis Follicularis

(1) Methods and Results

The hot water extract of Yokuinin (daily dose of 2 g of the extract ingredient) was administered to 15 female volunteers (18 to 26 years old) who were positive for keratosis follicularis. Observation was performed for at least 3 weeks, with 6 weeks as longest. Cases that had been diagnosed as treatment failure by the use of Yokuinin were subjected to this test. The enzyme extract of Coix seeds with husks (2 g/day of the extract)-administered group (7 cases, group A), the fermentation extract of Coix seeds with husks compounded with folic acid-administered group (10 mg/day of folic acid+2 g/day of the extract) (8 cases, group B) were observed for at least 3 weeks, with 6 weeks as longest. The results among the 7 cases of group A were: 1 case of complete response, 2 cases of partial response, and 4 cases of treatment failure. Further the results among the 8 cases of group B were: 5 cases of complete response, 1 case of partial response, and 2 cases of treatment failure.

Next, the following therapy was performed again for the above 6 cases of treatment failure at least 2 months after the above therapy. 3 cases of a group (group C) administered with Denshichi ginseng in addition to the fermentation extract of Coix seeds with husks compounded with folic acid (10 mg/day of folic acid+2 g/day of the extract+6 g/day of Denshichi ginseng), and 3 cases of a group (group D) administered with Denshichi ginseng alone were observed for 2 weeks. The results among group C were: 1 case of complete response, 2 cases of partial response and no case of treatment failure. On the other hand, the results among group D were: no case of complete response, 1 case of partial response, and 2 cases of treatment failure. In addition, no side effect was observed in any cases.

(2) Case Summary

Keratosis follicularis is a genetic dyskeratosis of hair follicles, and there is no therapy for this disease. Since keratosis follicularis is not a life-threatening disease, it is often left untreated. However, particularly in the case of young women, they are often nervous about beauty, and worry about, so-called shark skin (skin roughness) more than doctors imagine. Since the therapy by means of the fermentation extract of Coix seeds with husks also contributes to improve the QOL of patients, it is considered as an extremely usable therapy. Further, as a therapy for maintaining a case that has been improved by this therapy, it is recommended that the 2 g/day of the fermentation extract of Coix seeds with husks+400 μg/day of folic acid+3 g/day of Denshichi ginseng be administered continuously.

### EXAMPLE 15

### Keratosis Follicularis (a Case of Complete Response)

This example presents a case wherein a complete response was achieved against keratosis follicularis.

Case: 18 Years Old Woman

In her adolescence (14 years old), multiple non-inflammatory papulae were developed consistently at the pores of hair follicles on the extension sides of extremities, particularly at the upper arms and the skin of the shoulders. The patient visited a dermatologist, received therapy using an urea ointment and the like, but the symptoms did not improve. There were no symptoms such as itching and the like. However the skin gradually became shark-like skin and the lesions spread toward the extension side of the femoral region, she consulted a dermatology specialist. She was shocked by the diagnosis that this case was familial and there was no therapy for this case. Then she visited our out-patient clinic. Administration of Yokuinin for 3 weeks resulted in treatment failure. Thus with informed consent, the fermentation extract of Coix seeds with husks compounded with folic acid (2 g/day of the extract+10 mg/day of folic acid) was administered, skin roughness started to gradually disappear from 1 week after the administration, and it disappeared completely after 4 weeks. Further, black pigmentation that had been consistently observed at the hair follicles also disappeared, and a so-called whitening effect was confirmed all over the skin. This is clinically determined as a case of complete response.

### EXAMPLE 16

### Chloasma (Liver Spots)

Methods and Results

The hot water extract of Yokuinin (daily dose of 2 g of the extract ingredient) was administered to 22 female volunteers (30 to 66 years old) with Chloasma for 6 weeks. To cases that had been diagnosed as treatment failure, the enzyme extract of Coix seeds with husks (2 g/day of the extract) was administered for 6 weeks, after at least 2 months of non-medication period had been provided. The results were: 8 cases of complete disappearance of chloasma (complete response), 8 cases of improvement in chloasma (partial response) and 6 cases of treatment failure. Based on the above results, the enzyme extract of Coix seeds with husks is regarded as effective against chloasma.

### EXAMPLE 17

### Chloasma (a Case of Complete Response)

This example presents a case wherein a complete response was achieved by a compounding agent containing 3 agents.

(1) Case: 64 Years Old Female with Ovarian Cancer (Stage IIIa)

Approximately 5 years ago, 3×4 cm chloasmas developed on both sides of the face and a 1 cm verruca vulgaris developed on the left cheek, but these were left untreated. When an anticancer agent was administered this time, the enzyme extract of Coix seeds with husks (2 g/day)+folic acid (2 mg/day)+Denshichi ginseng (6 g/day) was administered everyday in anticipation of antineoplastic effect with informed consent. At the end of 2 courses of the anticancer agent (5 weeks after the administration), chloasmas on both sides of the face and the verruca vulgaris disappeared completely.

(2) Case Summary

Although the purpose of the administration of the enzyme extract of Coix seeds with husks was not for treating chloasma and verruca vulgaris in this case, chloasma and verruca vulgaris disappeared by chance. Based on the facts that chloasmas and verruca vulgaris had been continuously observed for about 5 years and normally, during administration of an anticancer agent, suppression of immunological functions and exacerbation of viral diseases are often found, this was diagnosed as a case wherein a complete response was achieved by the 3 agents including the enzyme extract of Coix seeds with husks. Moreover, this is a case wherein the whitening effect (an effect of preventing skin pigmentation) exerted on her face was confirmed by the testimony of the patient and her family members.

### EXAMPLE 18

### Freckle, Wrinkle, Senile Plaque, Skin Pigmentation, Clavus and Acne Vulgaris (Acne)

It has been shown that the fermentation extract of and the enzyme extract of Coix seeds with husks and a folic acid/Denshichi ginseng-compounded agent have an effect against freckles, wrinkles, senile plaques, skin pigmentation, clavus and acne vulgaris (acne). According to totalization of the cases that we observed, a complete response was achieved by the above drug in 26 cases with freckles, 14 cases with wrinkles (particularly on the face and extremities), 10 cases with senile plaque (particularly on the face), 51 cases with skin pigmentation, 4 cases with clavus, and 32 cases with acne vulgaris. In all the cases employed for totalization, clinical conditions had been continuously observed for at least 6 months. Further, the original purpose of the administration of the drug was not for treating these diseases, and the diseases were cured during the therapies against tumors and verrucae.

### EXAMPLE 19

### Actions to Relieve Constipation and to Eliminate the Smell of Stool

(1) Methods and Results

The hot water extract of Yokuinin (daily dose of 2 g of the extract ingredient) was administered for 1 week to 18 female volunteers and 14 male volunteers with habitual constipation characterized by the stool frequency of only once every 3 to 7 days. To 24 cases who had been diagnosed as treatment failure, 2 g/day of the fermentation extract of Coix seeds with husks was administered for 1 week, after at least a 2 weeks of non-medication period had been provided. The results were: 18 cases of improved Taxation (partial response), 3 cases of treatment failure and 3 cases were indeterminate. Among 24 cases, subjects of 16 cases became conscious of disappearance of the smell of stool. Further, none of the 32 cases became conscious of disappearance of the smell of stool while taking the hot water extract of Yokuinin.

(1) Case Summary

It was understood that the fermentation extract of Coix seeds with husks has excellent drug efficacy in capabilities of improving habitual constipation and eliminating the smell of stool, compared with the hot water extract of Yokuinin.

All publications, patents and patent applications cited herein are incorporated herein by reference in their entirety.

### INDUSTRIAL APPLICABILITY

According to the present invention, there is provided a composition for foods or medicaments, which contains the fermentation product or the enzyme-treated product of the husks, pellicles and astringent skin of Coix seeds, or the same compounded with folic acid or Denshichi ginseng. The composition of the present invention is useful as a preventive or a remedy against various diseases, or as a functional food.

1. A method for treatment of a tumor comprising administering to a patient presenting a tumor an effective amount of a composition comprising a fermentation product that is obtained by fermenting at least one type of component selected from the group consisting of husks, pellicles and astringent skin of Coix seeds, wherein the tumor is benign tumor or precancerous lesion.

2. The method according to claim 1, wherein the composition further comprises folic acid.

3. The method according to claim 1, wherein the benign tumor is a vocal cord polyp.

4. The method according to claim 1, wherein fermentation is performed by at least one type of microorganism selected from Aspergillus, lactic acid bacteria and yeast.

5. A method for treatment of a tumor comprising administering to a patient presenting the tumor an effective amount of a composition comprising an enzyme-treated product that is obtained by treating with an enzyme at least one type of component selected from the group consisting of husks, pellicles and astringent skin of Coix seeds, wherein the tumor is benign tumor or precancerous lesion.

6. The method according to claim 5, wherein the composition further comprises folic acid.

7. The method according to claim 5, wherein the benign tumor is a vocal cord polyp.

8. The method according to claim 5, wherein the enzyme is at least one type of agent selected from a diastase agent, a Takadiastase agent, an α-amylase agent, a β-amylase agent, a glucoamylase agent, a pectinase agent, a β-glucosidase agent, a cellulase agent, a hemicellulase agent and a xylanase agent.

9. A method for treatment of human papillomaviral diseases comprising administering to a patient presenting the human papillomaviral diseases an effective amount of a composition comprising a fermentation product that is obtained by fermenting at least one type of component selected from the group consisting of husks, pellicles and astringent skin of Coix seeds.

10. The method according to claim 9, wherein the composition further comprises folic acid.

11. The method according to claim 9, wherein the human papillomaviral disease is at least one type of disease selected from the group consisting of condyloma acuminatum, verruca vulgaris, adolescent verruca plana, senile verruca and laryngeal papillomatosis.

12. The method according to claim 9, wherein fermentation is performed by at least one type of microorganism selected from Aspergillus, lactic acid bacteria and yeast.

13. A method for treatment of human papillomaviral diseases comprising administering to a patient presenting the human papillomaviral diseases an effective amount of a composition comprising an enzyme-treated product that is obtained by treating with an enzyme at least one type of component selected from the group consisting of husks, pellicles and astringent skin of Coix seeds.

14. The method according to claim 13, wherein the composition further comprises folic acid.

15. The method according to claim 13, wherein the human papillomaviral disease is at least one type of disease selected from the group consisting of condyloma acuminatum, verruca vulgaris, adolescent verruca plana, senile verruca and laryngeal papillomatosis.

16. The method according to claim 13, wherein the enzyme is at least one type of agent selected from a diastase agent, a Takadiastase agent, an α-amylase agent, a β-amylase agent, a glucoamylase agent, a pectinase agent, a β-glucosidase agent, a cellulase agent, a hemicellulase agent and a xylanase agent.

17. A method for treatment of at least one type of condition selected from the group consisting of molluscum contagiosum, follicular keratosis, chloasma, freckle, wrinkle, senile plaque, skin pigmentation, skin roughness, clavus and acne vulgaris comprising administering to a patient presenting the condition an effective amount of a composition comprising a fermentation product that is obtained by fermenting at least one type of component selected from the group consisting of husks, pellicles and astringent skin of Coix seeds.

18. The method according to claim 17, wherein the composition further comprises folic acid.

19. The method according to claim 17, wherein fermentation is performed by at least one type of microorganism selected from Aspergillus, lactic acid bacteria and yeast.

20. A method for treatment of at least one type of condition selected from the group consisting of molluscum contagiosum, follicular keratosis, chloasma, freckle, wrinkle, senile plaque, skin pigmentation, skin roughness, clavus and acne vulgaris comprising administering to a patient presenting the condition an effective amount of a composition comprising an enzyme-treated product that is obtained by treating with an enzyme at least one type of component selected from the group consisting of husks, pellicles and astringent skin of Coix seeds.

21. The method according to claim 20, wherein the composition further comprises folic acid.

22. The method according to claim 20, wherein the enzyme is at least one type of agent selected from a diastase agent, a Takadiastase agent, an α-amylase agent, a β-amylase agent, a glucoamylase agent, a pectinase agent, a β-glucosidase agent, a cellulase agent, a hemicellulase agent and a xylanase agent.

23. A method for treatment of a tumor comprising administering to a patient presenting the tumor an effective amount of a composition comprising folic acid and a fermentation product that is obtained by fermenting at least one type of component selected from the group consisting of husks, pellicles and astringent skin of Coix seeds, wherein the tumor is at least one type of tumor selected from the group consisting of uterine cancer, vaginal cancer, vulvar cancer, skin cancer, esophageal cancer, oral cavity cancer, gingival cancer, jaw cancer, pharyngeal cancer, vocal cord carcinoma, lung cancer, bladder cancer, thyroid cancer, breast cancer, gastric cancer, colon cancer, pancreatic cancer, renal cancer, ovarian cancer, melanoma, a central nervous system tumor, a peripheral nervous system tumor, mediastinal tumor, hepatic cancer, bile duct cancer, gallbladder cancer, a renal pelvis tumor, ureter cancer, testicular tumor, prostate cancer, choriocarcinoma, fallopian tube cancer, sarcoma, leukemia, erythroleukemia, multiple myeloma, malignant lymphoma and carcinosarcoma.

24. The method according to claim 23, wherein fermentation is performed by at least one type of microorganism selected from Aspergillus, lactic acid bacteria and yeast.

25. A method for treatment of a tumor comprising administering to a patient presenting the tumor an effective amount of a composition comprising folic acid and an enzyme-treated product that is obtained by treating with an enzyme at least one type of component selected from the group consisting of husks, pellicles and astringent skin of Coix seeds, wherein the tumor is at least one type of tumor selected from the group consisting of uterine cancer, vaginal cancer, vulvar cancer, skin cancer, esophageal cancer, oral cavity cancer, gingival cancer, jaw cancer, pharyngeal cancer, vocal cord carcinoma, lung cancer, bladder cancer, thyroid cancer, breast cancer, gastric cancer, colon cancer, pancreatic cancer, renal cancer, ovarian cancer, melanoma, a central nervous system tumor, a peripheral nervous system tumor, mediastinal tumor, hepatic cancer, bile duct cancer, gallbladder cancer, a renal pelvis tumor, ureter cancer, testicular tumor, prostate cancer, choriocarcinoma, fallopian tube cancer, sarcoma, leukemia, erythroleukemia, multiple myeloma, malignant lymphoma and carcinosarcoma.

26. The method according to claim 25, wherein the enzyme is at least one type of agent selected from a diastase agent, a Takadiastase agent, an α-amylase agent, a β-amylase agent, a glucoamylase agent, a pectinase agent, a β-glucosidase agent, a cellulase agent, a hemicellulase agent and a xylanase agent.
